(19) United States
(12) Patent Application Publication
(10) Pub. No.: US 2015/0121561 A1
(43) Pub. Date: Apr. 30, 2015

(54) Method for Protein Production in Doubled Haploid Plants

(71) Applicant: Locusia OY, Helsinki (FI)

(72) Inventor: Kimmo Koivu, Helsinki (FI)

(73) Assignee: Locusia OY, Helsinki (FI)

(21) Appl. No.: 14/068,027

(22) Filed: Oct. 31, 2013

(51) Int. Cl. C12N 15/82

(52) U.S. Cl. CPC C12N 15/8257
USPC 800/260; 800/288; 435/410; 800/298

(57) ABSTRACT

A method to provide transgenic doubled haploid plants is provided. The doubled haploid plants of this disclosure express heterologous gene products in their seeds. The disclosure also provides a clonal-like isogenic production system in dicotyledonous plant seeds where transgene expression can be controlled. The disclosure specifically provides a system to produce high quality and quantity of heterologous gene product in doubled haploid Camelina sativa seeds.

### SEQUENCE LISTING

This application contains sequence listing which is provided on written format and on computer readable format which is identical to the written format.

### CLAIM OF PRIORITY

This application does not claim priority of any other patents or patent applications.

### FIELD OF INVENTION

This invention relates to a clonal-like isogenic production system in plant seeds. More specifically this invention relates to a clonal-like isogenic production system of recombinant proteins and modified seed oils in doubled haploid dicotyledonous plant seeds.

### BACKGROUND OF THE INVENTION

The chromosomes of higher plants exist in homologous pairs, and are called diploids. Gametic cells contain half of the normal number of chromosomes and are said to be haploid. When two gametic cells unite, the two sets of haploid chromosomes combine to create a set of diploid chromosomes. A doubled haploid plant is formed when the chromosomes of a haploid cell are doubled such that each chromosome in a homologous pair is identical.

Doubled haploid-methodology is commonly used in agronomically important crops, such as corn, to speed the development of new cultivars. With traditional methods it takes about 10 years to develop a new cultivar, while with doubled haploid-technology the breeding period can be reduced to about third. Haploid plants are commonly produced using one of four methods: a) culture of anthers or microspores (androgenesis), b) culture of unfertilized ovules (gynogenesis), c) interspecific or intergeneric crosses followed by chromosome elimination, and d) pollination with irradiated pollen. In many species the culture of isolated microspores or anthers is the most frequently used method of haploid production.

Genetic engineering is an essential part of plant breeding today. Haploid systems are commonly used for microspore mutagenesis. The haploid system is well suited for genetic transformation as well. For example US2008/0216198 provides a method to transform haploid embryos of Zea mays and then treat the transformed embryo cells with chromosome doubling agent. US2009/0293141 discloses Agrobacterium-transformation of immature embryos of a double hybrid corn line.

There are also examples of transgenic plants produced by transforming microspores or microspore originated callus and then cultivating the transgenic haploid plants. For example U.S. Pat. No. 7,297,838 discloses creating an anther culture of flax-plants (Linum usitatissimum) and transforming the anther-culture obtained callus with Agrobacterium harboring beta-glucuronidase encoding gus-gene.

However, the examples of transforming double haploid plants are scarce. Tsukazaki et al. 2002 discloses transformation of hypocotyls of doubled haploid Brassica oleracea plants. The hypocotyls were transformed with beta glucuronidase encoding gus-gene and the gene expression was tested of the hypocotyl or leaf segments. The best transformation rate disclosed in this publication was 3.1% (3 transformants per 98 explants).

Kazan et al. 1997 dislcose Agrobacterium-mediated transformation of doubled haploid Brassica napus lines. The transformation was conducted with hypocotyl segments and roots as explants. The Agrobacterium strains harbored vectors containing GUS encoding sequences and GUS-expression was detected in petals, leaves, stamens and anthers of the transformed doubled haploid lines. Similarly as in Tsukazaki the transformation rates were extremely low and in many conditions no transformants were obtained.

Cross pollination rates of Brassica napus can be 20-30%, which would create difficulties to maintain a doubled haploid line from generation to generation. Therefore, the doubled haploid Brassica lines of Tsukazaki et al and Kazan et al may be suitable mainly for research purposes as new lines would need to be generated at time to time.

Accordingly, there are methods to produce doubled haploid lines from a number of plant species. However, the critical step is managing to produce the microspore cultivation and the conditions are highly dependent on the plant species. There are a number of factors affecting microspore embryogenesis including genotype, donor plant growth conditions, stage of microspore development, composition of the culture medium, and environmental conditions during culture. The frequency of embryo production will depend on whether or not these conditions are optimal and varies depending on the species. Furthermore, in order to maintain the doubled haploid mother lines the species has to be selected with the cross pollination frequency in mind.

Once the microspore cultivation is established for a species, it is relatively common to use the microspore originated cells or embryos for mutagenesis or genetic transformation. However, it is not a common practice to grow double haploid plants and use these plants as explants for genetic transformation. This approach has been taken by Tzukazaki et al. and by Kazan et al. to transform Brassica double haploid plants with GUS-gene expressing in plant shoots. However, they were able to obtain only extremely low transformation rates.

Ferrie and Bethune 2011 have published a protocol for microspore embryogenesis for Camelina sativa. However, there is no record of anyone having used doubled haploid Camelina sativa plants as explants for gene transformation. Neither do we have any record of anyone having transformed double haploid dicotyledonous plants to express heterologous proteins in their seeds.

There is a constant need for a system to produce large quantities of uniform quality of heterologous proteins and other recombinant gene products for industrial and pharmaceutical uses.

Additionally there is a need for a production system where the expression level of heterologous products could be controlled.

This invention will provide solutions to these needs and others not provided by currently known art.

### SUMMARY

It is an object of this invention to provide doubled haploid lines of Camelina sativa plants.

It is another object of this invention to provide a transformation system for doubled haploid Camelina sativa plants.

It is yet another object of this invention to provide a method to introduce seed expressing heterologous genes into doubled haploid dicotyledonous plants.

Yet another object of this invention is to provide a method to produce heterologous proteins in doubled haploid dicotyledonous plant seeds for industrial and pharmacological uses.

It is an object of this invention to provide a clonal-like isogenic production system in dicotyledonous plant seeds to produce heterologous proteins and modified seed oils.

It is still another object of this invention to provide a clonal-like isogenic production system in dicotyledonous plant seeds to produce heterologous proteins in large quantities with high uniformity of the product.

It is another object of this invention to provide a clonal-like isogenic production system in dicotyledonous plant seeds to produce altered seed oils in large quantities with high uniformity of the product.

It is an object of this invention to provide a clonal-like isogenic production system where the expression level of heterologous gene product can be controlled by manipulating the copy number of the transgenes in doubled haploid plant material.

A further object of this invention is to provide a stable double haploid clonal-like isogenic Camelina sativa mother seed line, the seeds of which are germinated and used as an explant source for Agrobacterium mediated transformations.

Still another object of this invention is to provide a stable double haploid clonal-like isogenic Camelina sativa mother seed line, the seeds of which are germinated and grown to mature flowering plants that are used for Agrobacterium mediated flower dip transformations.

A further object of this invention is to provide a transgenic double haploid seed of Camelina sativa plants.

It is an object of this invention to provide a method to produce one or more heterologous gene products in dicotyledonous plant seeds, said method comprising the steps of:

a) providing a haploid cell culture;

b) obtaining haploid embryos or callus from the haploid cell culture;

c) regenerating the embryos or the callus to double haploid plantlets with shoots and roots;

d) obtaining double haploid plants from the doubled haploid plantlets;

e) obtaining double haploid seeds from the mature double haploid plants;

f) surface sterilizing the double haploid seeds;

g) germinating the seed of step f) in vitro to form doubled haploid plantlets or sprouts;

h) transforming explants from the double haploid plantlets or sprouts with Agrobacterium tumefaciens carrying at least one vector comprising a nucleotide sequence encoding the heterologous gene product under a seed specific promoter;

i) regenerating the transformed explants to transgenic doubled haploid plants; and

j) allowing the plants to produce seeds expressing the heterologous gene product.

According to preferred embodiment the plant species may be Camelina sativa, the seed specific promoter may be napin promoter and the heterologous gene product may be selected from the group consisting of lauric acid-acyl carrier protein, hydrophobin, tropoelastin, an antibody, and a growth factor.

According to a preferred embodiment the tropoelastin is encoded by a synthetic gene having nucleotide sequence according to SEQ ID NO: 5.

It is another object of this invention to provide a clonal-like isogenic production system in dicotyledonous plant seeds to produce heterologous gene products in large quantities with high uniformity of the product, said system comprising:

a) a haploid cell culture to provide haploid callus or embryos;

b) a protocol to regenerate the haploid callus or embryos to doubled haploid plantlets;

c) a protocol to grow doubled haploid plants from the plantlets of step b);

d) a protocol to produce double haploid seeds of plants of step c);

e) a protocol to produce explants from the double haploid seeds of step d)

f) a protocol to transform a nucleotide sequence encoding the heterologous gene product under a seed specific promoter to the explants of step e);

g) a protocol to regenerate the explants to doubled haploid transgenic plants;

h) a protocol to grow the doubled haploid transgenic plants to maturity, and to collect doubled haploid transgenic seeds of the mature plants; and

g) a protocol to harvest the heterologous gene products from the transgenic seeds.

According to one preferred embodiment the clonal-like isogenic production system is provided in Camelina sativa seeds.

It is still another object of this invention to provide a method to control production of heterologous gene products in dicotyledonous plant seeds, said method comprising the steps of:

a) providing a haploid cell culture;
b) obtaining haploid embryos or callus from the haploid cell culture;
c) regenerating the embryos or the callus to double haploid plantlets with shoots and roots;
d) obtaining double haploid plants
e) obtaining double haploid seeds from the double haploid plants
f) surface sterilizing the seed of step e)
g) germinating the seeds of step f) in vitro
h) transforming explants from double haploid plantlets or sprouts with Agrobacterium tumefaciens carrying at least one vector comprising a nucleotide sequence encoding a nucleotide sequence encoding the heterologous gene product under a seed specific promoter;
i) regenerating the transformed explants to transgenic doubled haploid plants;
j) determining copy number of the nucleotide sequence encoding the heterologous gene product in several doubled haploid plants;
k) selecting lines with preferred copy numbers, and
l) allowing the selected lines to produce flowers and crossing the lines, thereby receiving next generation with a increased copy number and desired level of production of the heterologic gene product.

It is still another object of this invention to provide a transgenic doubled haploid seed of Camelina sativa expressing a heterologous gene product.

According to preferred embodiments the heterologous gene product may be expressed under napin promoter.

According to preferred embodiment the plant species may be Camelina sativa, the seed specific promoter may be napin promoter and the heterologous gene product may be selected from the group consisting of lauric acid-acyl carrier protein, hydrophobin, tropoelastin, an antibody, and a growth factor.

### SHORT DESCRIPTION OF THE DRAWINGS

FIG. 1 Expression cassette for seed specific production for Shitzophyllum commune hydrophobin SC3, constructed by PCR overlap.

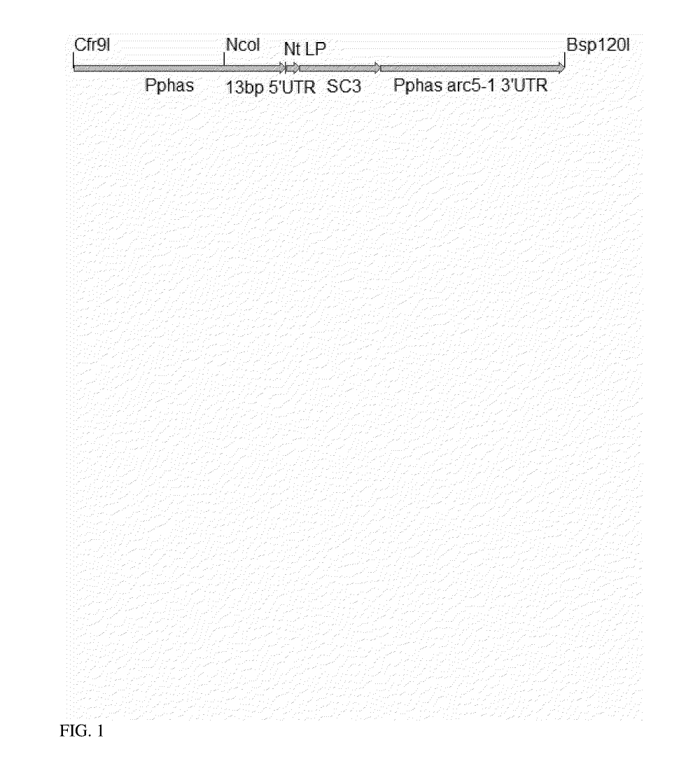

### DETAILED DESCRIPTION OF THE INVENTION

### Definitions

Doubled haploid means a plant or plant tissue generated from a haploid cell, where each cell has two identical sets of haploid chromosomes.

Hydrophobin proteins means monomeric or multimeric hydrophobin proteins.

Stably transformed plant means a transgenic plant where the transgenic material is inserted into the genome of the plant, whereby the transgenic character is inherited according to Mendelian rules.

A protocol to regenerate the haploid callus or embryos to doubled haploid plants means methods as described in Example 4 for Camelina sativa plants. The protocol described in Example 4 may be modified for other plant species and such modifications are also encompassed by the term ‘protocol to regenerate the haploid callus or embryos to doubled haploid plants’.

A protocol to transform a nucleotide sequence encoding the heterologous gene product under a seed specific promoter to explants obtained from the doubled haploid plants means a transformation method of a nucleotide sequence encoding a heterologous gene product under a seed specific promoter to explants as is described in Example 6 for Camelina sativa. The transformation is preferably Agrobacterium mediated transformation but other transformation methods may also be used.

A protocol to produce explants from the double haploid seeds means methods as described in Example 6 for Camelina sativa plants. The protocol described in example 6 may be modified for other plant species and such modifications are also encompassed by this term.

A protocol to regenerate the explants to doubled haploid transgenic plants means methods to regenerate the transgenic explants to doubled haploid transgenic plants as described in Example 6 for Camelina sativa. The method may be modified for other plant species and such modifications are encompassed by the term “a protocol to regenerate the explants to doubled haploid transgenic plants”.

A protocol to grow the doubled haploid transgenic plants to maturity, and to collect doubled haploid transgenic seeds of the mature plants means methods such as described in Example 10 for Camelina sativa to grow the plants, to let them form inflorescence and produce seeds and finally collect the seeds from the plants. The plants may be grown in growth chambers, green house or in field conditions in a controlled manner.

A protocol to harvest the heterologous gene products from the transgenic seeds means method such as described in Examples 7 and 8. The protocol may vary depending on the gene product and such variations are encompassed by the term.

Copy number means the number of heterologous DNA inserts in a genome.

Haploid cell culture is an anther culture, a pollen culture, a microspore culture or an ovule culture.

Anther Culture: Anther culture is technique by which the developing anthers at a precise and critical stage are excised aseptically from unopened flower bud and are cultured on a nutrient medium where the microspore within the cultured anther develop into callus tissue or embryoids that give rise to haploid plantlets either through organogenesis or embryogenesis.

Pollen or Microspore Culture: Pollen or microspore culture is an in vitro technique by which the pollen grains, preferably at the uninucleated stage, are squeezed out aseptically from the intact anther and then cultured on nutrient medium where the microscope, without producing male gametes, develop into haploid embryoids or callus tissues that give rise to haploid plantlets by embryogenesis or organogenesis.

Ovule Culture: Ovule culture is an experimental system by which ovules are aseptically isolated from the ovary and are grown aseptically on chemically defined nutrient medium under controlled conditions.

Allopolyploidy is a common genetic status of several plants: the plant has more than two sets of diploid chromosomes in its genome.

Camelina sativa is an allohexaploidic species and it has actually three sets of diploid chromosomes originating from ancient crossings of three relative species. The allohexaploid nature of the genome of Camelina sativa may have multiple implications. Polyploids are generally thought to be more adaptable to changing environments and more flexible in occupying larger niches than any one of their ancestors. On genetic level, recessive mutations in a hexaploid genome could be masked by redundant homologous loci. Allohexaploidy increases the diversity and genetic variation between generations. In transgenic allopolyploidic plants the multiple sets of diploid chromosomes will affect quantity and quality of recombinant protein. Therefore, allohexyloidic Camelina sativa as a target for production of recombinant gene products may not provide an optimal result.

In this disclosure, it is shown how recombinant gene products can be produced in high and uniform quality in seeds of double haploid Camelina sativa plants.

In this disclosure a clonal production system in plants is provided. The production system is provided in doubled haploid seeds. Camelina sativa is a preferred species for the method disclosed in this disclosure due to its high frequency of self pollination (practically 100%).

The same concept may be applied to several other dicotylenous plant species, such as Nicotiana tabacum, Brassica sp., Medigago sativa, sunflower, safflower, soybean and alfalfa. However, these plant species may require generation and selection of new mother lines due to cross pollination as opposed to primarily self pollinating Camelina. Brassica oleracea is a vegetable plant and mainly cross pollinating and for this reason not suitable for seed based protein production.

Generally the production system in doubled haploid seeds can be established to produce heterologous proteins for industrial and pharmaceutical uses. Examples of industrial proteins to be produced in the clonal production system of this disclosure include but are not limited to hydrophobins, and tropoelastins. Examples of pharmaceutical proteins to be produced in the clonal production system of this disclosure include but are not limited to growth factors and recombinant antibodies. The clonal-like isogenic production system of this invention is also suitable to produce modified seed oils for human nutrition or industrial purposes. An examples of modified oils for human nutrition is long chained polyunsaturated fatty acids.

Specifically this disclosure provides constructs to express heterologous gene products in seeds. Examples of plant promoters and terminators directing the expression into the seeds, are napin-promoter and -terminator.

According to a preferred embodiment of this invention a doubled haploid line of Camelina sativa plant regenerated from microspore culture. The doubled haploid plants are grown in green house and surface sterilized seeds of the plants are germinated in vitro and used as explants for Agrobacterium transformation. The transformed doubled haploid explants are regenerated to shoots and roots and transferred to green house conditions. The transgenic doubled haploid plants are let to bloom and self pollinate and the transgenic seeds are used for extraction of the recombinant gene product.

According to one preferred embodiment the doubled haploid plants are transformed with Agrobacterium harboring a transformation vector with Scitzophyllum communune SC3 hydrophobin encoding sequences under seed specific promoter.

According to another preferred embodiment the doubled haploid plants are transformed with Agrobacterium harboring a transformation vector with human tropoelasting endocing sequences under seed specific promoter.

According to yet another preferred embodiment the doubled haploid plants are transformed with Agrobacterium harboring a transformation vector harboring California bay laureate carrier protein under a seed specific promoter.

Hydrophopins are small surface-active multipurpose proteins that are used in cell culture, nanotechnology, pharmacy, cosmetics and food production. The current art knows production of monomeric hydrophobin in E. coli. Also transient expression of hydrophobin as part of a fusion protein has been described (Joensuu et al. 2010). A method to stably transform pant tissue to express hydrophobin has been disclosed by the inventor if this disclosure in US 2013/0219559. However, transformation of and expression in double haploid plant material with monomeric or multimeric hydrophobins has never been shown.

Tropoelastin is used for cell culture scaffolds, artificial vein and skin crafts, cosmetics among a variety of other purposes. The current art knows expression of human tropoelastin in E. coli transformation system. The full natural tropoelastin protein sequence has not been introduced into the plant expression systems and specifically never to doubled haploid plants or doubled haploid seeds.

Need for recombinant antibodies and their derivatives increases continuously in diverse applications. There are various disclosures showing expression of recombinant antibodies is plant tissue, such as tobacco leaves, but doubled haploid plants have never been transformed with antibody encoding genes.

The invention is now described by means of nonlimiting examples. A skilled artisan would recognize that various modifications to the examples may be made without diverting from the spirit of the invention.

### Example 1

### Cloning the Expression Vector for Production of Hydrophobin in the Seeds of Double Haploid Camelina Sativa

Seed specific expression cassette with Phaseolus vulgaris phaseolin promoter sequence, arcelin 5′ UTR and arcelin 3′ UTR was constructed as follows.

Genomic DNA for PCR amplification of Phaseolus vulgaris control elements is extracted from germinated seedlings with standard extraction methods. Alternatively, a kit such as Fermentas GeneJet Genomic DNA extraction kit is used. Phaseolus vulgaris phaseolin promoter sequence (Genebank accession number J01263; SEQ ID NO:1) is amplified with primers o1L (SEQ ID NO:10) and o13 (SEQ ID NO:11) from genomic DNA of Phaseolus vulgaris to correspond nucleotides 1 to 1470 of the promoter sequence. Short homology stretches with vector backbone containing Cfr9I restriction site at the 5′ end and with Phaseolus vulgaris arcelin 5′ UTR and expressed gene of interest at the 3′ end are introduced into the sequence with PCR primers. Synthetic gene sequence with Nicotiana tabacum leader peptide and Scizophyllum commune SC3 (SEQ ID NO: 2 for synthetic gene, SEQ ID NO: 3 for amino acid sequence) is amplified with primers o11 (SEQ ID NO:24) and o12 (SEQ ID NO:25) simultaneously introducing short overlaps with amplified Phaseolus vulgaris arcelin 5′UTR sequence (SEQ ID NO:26) and arcelin 3′UTR sequence (Genebank accession number Z50202 to correspond nucleotides 2621-3900, (SEQ ID NO:4). Arcelin 3′UTR is bought as synthetic DNA and amplified with primers o5 (SEQ ID NO:12) and o6L (SEQ ID NO:13), simultaneously introducing short overlap with vector backbone, containing Bsp120I restriction site. All PCR reactions are constructed according to standard protocols. High Fidelity Phusion polymerase (Thermo Lab system) is used for the amplification. PCR products are combined to seed specific expression cassette by separate overlap PCR reaction and cloned into plant expression vector with Cfr9I/Bsp120I.

### Example 2

### Cloning the Expression Vector for Production of Tropoelastin in the Seeds of Double Haploid Camelina Sativa

Synthetic sequence for part of Arabidopsis thaliana seed storage albumin 2 signal peptide (2S2 SP) coded by nucleotides 80-118 of Genebank accession number NM_{—}118849 and human tropoelastin (ELN) coding for amino acid sequence coded by nucleotides 170-2167 of Genebank accession number NM_{—}001081753 (SEQ ID NO: 5 for synthetic gene, SEQ ID NO:6 for amino acid sequence) is obtained from GeneScript. The 2S2-ELN is amplified by PCR from GeneScripts vector pUC57 using primers o115 (SEQ ID NO: 14) and o117 (SEQ ID NO:15). 6× His-KDEL-Stop-fragment is made by PCR using two overlapping primers o116 (SEQ ID NO:16) and o118 (SEQ ID NO:17). 2S2-ELN and 6× His-KDEL-Stop-fragment have homologous overlap and are joined together in second PCR using primers o115 (SEQ ID NO:14) and o118 (SEQ ID NO:17). In addition to homology to ELN, 6× His-KDEL-Stop-fragment contain homology to arcelin 3′UTR.

Plant expression vector pA14-PPhas-2S2-Tarc-2-3 contain Phaseolus vulgaris phaseolin promoter (SEQ ID NO:1), Arabidopsis thaliana seed storage albumin 2 signal peptide (2S2 SP) (SEQ ID NO:10) and arcelin 3′UTR (SEQ ID NO:4). pA14-PPhas-2S2-Tarc-2-3 is cut with restriction enzymes XhoI and Paul between the 2S2 SP and arcselin 3′UTR and joined via end-homologies with 2S2-ELN-6× His-KDEL-Stop fragment using Gibson Assembly reaction mix of New England Biolabs.

### Example 3

### Cloning of the Expression Vector Targeting to Metabolic Engineering of Double Haploid Camelina Sativa

Agrobacterium Mediated Transformation with Lauric Acid-Acyl Carrier Protein (ACP) (EC 3.1.2.21-dodecanoyl-(acyl-carrier-protein)hydrolase) from California Bay (Umbellularia Californica)

Seed specific expression cassette with Brassica napus napin-A promoter sequence, with UcFatB1: lauric acid-acyl carrier protein (ACP) (EC 3.1.2.21-dodecanoyl-(acyl-carrier-protein)hydrolase) from California bay plant (Umbellularia californica) and UcFatB1 3′ UTR was constructed as follows: Genomic DNA for PCR amplification of Brassica napus napin-A promoter sequence is extracted from germinated seedlings with standard extraction methods. Alternatively, a kit such as Fermentas GeneJet Genomic DNA extraction kit is used. Brassica napus napin-A promoter sequence (Genebank accession number J02798; SEQ ID NO:7) is amplified with primers P-Bn-napA-F (SEQ ID NO:18) and P-Bn-napA-R (SEQ ID NO:19) from genomic DNA of Brassica napus) to correspond nucleotides 1 to 1145 of the J02798 sequence. The 5′ end of the forward primer contain XmaI restriction site. The 5′ end of the reverse primer contain XmaI and BpiI restriction sites. The product is digested with XmaI and cloned into pGem7 vector, which is cut with XmaI restriction enzyme and dephosphorylated with CIP to make pGem7-P-Bn-napA. A synthetic construct containing UcFatB1 to correspond nucleotides 145 to 1293 of Genebank accession number M94159 sequence and UcFatB1 3′UTR to correspond nucleotides 1294 to 1561 of Genebank accession number M94159 sequence and flanking restriction sites for BpiI and Bsp120I (SEQ ID NO:8) is obtained from synthetic DNA supplier. The UcFatB1-3′UTR fragment is cut out from the suppliers vector using BpiI and Bsp120I and cloned into pGem7-P-Bn-napA which is cut with the same restriction enzymes and dephosphorylated with CIP to make pGem7-P-Bn-napA-UcFatB1.

The expression cassette is cut from pGem7-P-Bn-napA-UcFatB1 using XmaI and Bsp120I restriction enzymes and cloned into pA5-MCS plant expression vector which is cut with same restriction enzymes and dephosphorylated with CIP to make construct pL1.

### Example 4

### Generation of Doubled Haploid Mother Plant Lines

Three Camelina sativa cultivars: Calena, Celine and Blaine Greek are subjected to microspore culture.

Donor plants are cultivated in greenhouse-like conditions, in 15 cm pots filled with peat-vermiculite mixture. Plants are watered according to need (estimated on daily basis) with tap water supplemented with mild fertilizer mixture. Soon after the inflorescences started to develop, they are removed from the plants. Small buds (˜1-3 mm) are selected to be used to set up the microspore culture.

Buds are initially briefly rinsed with 70% ethanol and subsequently rinsed with sterile, double-distilled water to completely remove the ethanol. The rinsed buds are then subjected to sterilization by incubating them for 10-15 min in 6% sodium hypochlorite, followed by several rinses (at least 3×5 min) with sterile, double distilled water.

Sterilized buds are homogenized either with a Warren blender or compatible homogenisator and suspended to ½× Gamborg G5 medium supplemented with 13% sucrose, pH 5.8. Crude suspensions are filtered through a sterile funnel lined with a nylon screen cloth with cut-off of 41 p.m. Crude microspore suspensions are then centrifuged at 130-150×g for 3 min, supernatants are decanted and pellets are resuspended to fresh NLN-medium supplemented with 12.5% sucrose and 12.5% PEG4000, without glutamine, pH 5.8. Pelleting and resuspending is repeated in total of three times. The number of microspores in the medium is determined with a Burker chamber or a corresponding cell counter and the medium is adjusted to have 10,000-25,000 microspores per ml.

10 ml of microspore solution is dispensed on 100×15 mm Petri plates. Plates are incubated in the dark, +24° C. for 72 h and supplemented with glutamine with final concentration of 0.8% w/v. Incubation in the dark is continued for 28 days. Embryos are then transferred to light, shaking at 50 rpm, with 16 h photoperiod. After about 5-10 days in the light cycle, the embryos are plated onto solid ½× Gamborg medium supplemented with 1% sucrose, 0.8% agar, pH 5.8. After 6-8 weeks of incubation, plantlets with well regenerated shoots and roots are transferred to a greenhouse and cultivated in peat-vermiculite mix until maturation. Production of fertile flowers under strictly self-pollinating conditions is considered as an evidence of successful doubled haploid generation.

### Example 5

### Screening Doubled Haploid Plants for Selection of Suitable Mother Plants for Transformation

Microspore cultivation is established using three different Camelina sativa varieties (Calena, Celine and Blaine Greek). Because different varieties respond differently in microspore cultivation two best varieties are selected based on the success of the cultivation. From both of the selected microspores cultures 100 healthy looking plantlets are chosen and the plantlets are transferred into soil and grown in a green house. The plants are grown in 16/8 hours light/dark cycle at 25/18 C° degrees. The plants are watered with 0.15% 20-20-20 fertilizer as needed. Of both of the varieties 25 healthiest looking plants are selected and the chromosome number is determined in order to avoid selecting mutated or aneuploidic individuals. 10 plants of each variety, showing double haploid chromosomes are selected for further screening.

The selected 10 plants are allowed to flower, self pollinate and produce seeds. The generation thus obtained is screened first for morphological characters, including leaf area, root weight, and number of inflorescence and seeds per inflorescence. The plants are also screened by their physiological characters, such as photosynthetic efficiency, drought tolerance, seed oil content, seed protein content.

Based on these characters 5 mother plants are selected from each cultivar. The selected doubled haploid mother plants thus represent plants with highest productivity, preferably best drought tolerance and optimal capacity to be donor plants for microspore culture.

### Example 6

### Transformation of Doubled Haploid Camelina Sativa Mother Plant Lines

The seeds of doubled haploid Camelina sativa mother plant are sterilized by immersing in 70% ethanol for 1 min and then treating for 5 min with Na-hypochlorite solution (2% active Cl⁻) with an addition of Tween-20 (1 drop per 100 ml). After sterilization the seeds are washed four times in sterile water and placed on solid Murashige and Skoog (MS) agar medium (Murashige and Skoog, Physiol. Plant. 15:472-493, 1962) without sugars for germination. Sterilized seeds are germinated and grown for two weeks on solid Murashige and Skoog (MS) medium without hormones. First true leaves serve as a source of explants for transformation procedure.

Agrobacterium tumefaciens-strain C58 carrying plant transformation vectors as describe above are grown overnight at 28° C. in liquid YEB medium (Lichtenstein and Draper, Gene Engineering of Plants. In: Glover DM (ed.) DNA Cloning—A Practical Approach, vol. 2. Oxford IRL, Oxford, pp 67-119, 1985) supplemented with 50 mg/l kanamycin and 10 mg/l rifampicin. Agrobacterium-culture of OD.sub.600=0.5 is used in the transformation experiments.

Leaves of in vitro grown doubled haploid Camelina sativa plants are cut with sharp knife blade on sterile wet tissue paper containing diluted overnight culture of Agrobacterium-solution. Leaves are cut into two halves across the leaf blade. The leaf segments with Agrobacterium are cultivated for 48 hours on Murashige and Skoog (MS) 0.7% agar medium supplemented with 1 mg/l 6-benzylaminopurine (BAP) and 0.3 mg/l alpha-naphthaleneacetic acid (NAA).

All the Murashige and Skoog (MS) culture media are supplemented with 1% sucrose and all in vitro cultures are kept at temperatures of 25° C. (day) and 18° C. (night) under the photoperiod of 16 h.

The explants are washed with water containing 300 mg/l ticarcillin. The explants are placed on Murashige and Skoog (MS) medium supplemented with hormones [0.7 mg/l 6-benzylaminopurine (BAP), 0.3 mg/l alpha-naphthaleneacetic acid (NAA)] and 200 mg/l ticarcillin and 0.05 mg/L imidazole. Two to three weeks old shoots are placed to the normal or half strength Murashige and Skoog (MS) medium solidified with 0.7% agar and supplemented with 200 mg/l Ticarcillin and optionally 0.1 mg/L imidazole and 0.3 mg/l alpha-naphthaleneacetic acid (NAA). Rooted shoots are transferred to soil and transgenic doubled haploid plants are grown in greenhouse or comparable conditions.

The doubled haploid plants are allowed to bloom and produce seeds. The recombinant gene expression in the transgenic doubled haploid seeds is confirmed with a northern blot analysis and the presence of the transgene is confirmed with Southern analysis.

Transformation efficiency is surprisingly high, 50-80% of explants express GUS reporter gene one week after transformation. Also the transgenic shoot regeneration efficiency is high, being 5-20% of explants. The total soluble protein of the seeds from the transformed plants varies between 0.1 and 10%. The glycosylation structure of the proteins produced in the doubled haploid seeds has more constant glycosylation structure and less variation in microheterogeneity in polypeptide sequence than proteins produced in transformed wild type plants. This is due to the minimal genetic variation in meiosis of the doubled haploid plants as the doubled haploid genome is fully homozygotic.

It is known that genomic insertion site or -position of the transgene may have major effect for transgene expression. Stable genetic background of the doubled haploid plants in subsequent generations reduces position effect because position effect is dependent on genomic background and if genetic background changes also position effect changes. Reduced position effect reduce batch-to -batch variation and makes the product more constant generation after generation.

Instead of the above described transformation method, the doubled haploid mother plants may be transformed by flower dipping method as described in U.S. patent application Ser. No. 12/933,827. According to this method Agrobacterium-mediated procedure comprises dipping flower clusters of doubled haploid Camelina sativa plants into a solution having an Agrobacterium comprising the heterologous gene sequence under the seed specific promoter as described above, a sugar and a nonionic surfactant. The method may include more than one dipping cycle, wherein a dipping cycle includes at least one step where the plant is dipped into the solution. As an alternative, the methods of the present invention can also comprise spraying the dipping solution onto the floral clusters of the doubled haploid plants or onto the sprouted seeds of the doubled haploid plants.

### Example 7

### Extraction of Hydrophobin from the Double Haploid Camelina Sativa Seeds

0.5 g of freshly harvested doubled haploid Camelina sativa seeds expressing hydrophobin were put into mortar and covered with liquid nitrogen. Seeds were ground to fine powder. Mortar was set on ice and the powder was let to let to thaw and 15 ml PBS (Phosphate-buffered saline extraction buffer: 137 mM CaCl2, 2.7 mM KCl, 8.1 mM Na2HPO4 and 1.8 mM KH2PO4; pH 7) was added. Extract was transferred to test tube and centrifuged 5 minutes at 20 000 at 4° C. The supernatant was collected into a new tube.

400 mg of triton X 114 was weighed into the 15 mil test tube. 10 ml of seed extract was preheated in 24° C. water bath for 5 min and seed extract/Triton X114 mixture was vortexed thoroughly for 1 min. The tube was set back to 24° C. water bath for 20 minutes to separate the phases.

The lower phase containing hydrophobic was transferred by pipetting, starting from the bottom of the tube, to a new tube. 4 ml isobutanol was added to hydrophobin containing lower phase and mixed well. The tube was set to a 24° C. water bath to separate phase. The lower phase containing the hydrophobin was collected to a new test tube. Isobutanol was removed by filtration though Biogel P-6 column (Bio-Rad). Hydrophobin containing solution was stored at 4° C. temperature. SDS-PAGE analysis was further used to analyze the hydrophobin contents of the solution. The total hydrophobin content of the seeds varies between 0.1 and 10%.

### Example 8

### Extraction of Tropoelastin from Doubled Haploid Transgenic Camelina Sativa Seeds

One gram of tropoelastin expressing seeds of C. sativa is homogenized in liquid nitrogen and frozen powder is transferred to 50 ml test tube containing 40 ml extraction buffer containing 100 mM Tris-HCL, pH 8.0, 150 mM NaCl and 0.5% triton X-100. Extract is filtrated trough Miracloth and centrifuged 20 000 g 10 min+4° C. Supernatant is used to isolate His-taged tropoelastin using HIS-select Nickel affinity gel. Column containing 1 ml HIS-select Nickel affinity gel is equilibrated and washed using 50 mM sodium phosphate buffer, pH 8.0 with 0.3 M NaCl and 10 mM imidazole. Plant extract is applied to gravity flow column and washed with 10 ml washing buffer. Tropoelastin is eluted using two ml 50 mM sodium phosphate, pH 8.0 with 0.3 M NaCl and 250 mM imidazole. Tropoelastin content of the seeds is about 100 μg per 1 g of seeds using micro-BCA method according to manufactures (Pierce) protocol.

### Example 9

### Extraction of Modified Oil from the Transgenic Doubled Haploid Seeds

Commercial service is used to analyze fatty acid profile and seed oil content of the transgenic doubled haploid Camelina sativa seeds. Total seed oil content is comparable to that of doubled haploid mother plant and lauric acid (12:0) content is 25% and oleic acid content is degreased from 17% to 10%.

### Example 10

### Upscale Production System for Producing Recombinant Proteins or Modified Oils in Doubled Haploid Camelina Sativa Plants

The selected mother lines are cultivated in green house like conditions and seed production of starting material for genetic transformation is thus ensured. The seeds are collected, germinated and explant material for transformation is obtained as described above. The explants are transformed with Agrobacterium harbouring at least one nucleotide sequence under seed specific promoter encoding a desired gene product. The transformed explants are regenerated as described above to plantlets that are transferred to green house like conditions. The production level of hydrophobin in doubled haploid Camelina seeds is within a range of 0.5-50 grams of hydrophobin or tropoelastin per kilogram of seeds. Accordingly a green house of 3000 square meters produces in average 2000 kg of Camelina sativa seeds and at a production of 25 grams per kilogram of seeds the greenhouse would produce 50 kg of hydrophobin or tropoelastin.

According to another embodiment the seeds of the doubled haploid plants are collected as above, but the seeds are transferred into sprouting facility where the seeds are allowed to sprout, the sprouted seeds are then collected, smashed and the heterologous product is collected from the smashed seed/spout mass.

### Example 11

### Determination of Copy Number of Transgenes Using Southern Blot Analysis

T-DNA copy number is determined using Southern blot analysis. Digoxigenin-11-UTP labeled probes homologous to transgene are synthesized. Genomic DNA of the transgenic plants are digested using separately restriction enzymes, which have recognition site in T-DNA only other side of the probe sequences. Plants, which produce a single positive band using two or more restriction enzymes, are considered to carry only one copy of the T-DNA.

After crossing two single copy plants, portion of the offspring's should produce hybridizing pattern of both parents. Total genomic DNA is isolated from leaf tissue of Camelina sativa plants using DNeasy Plant Midi Kit according to the supplier's instructions (Qiagen). Three micrograms of DNA is digested with restriction enzymes. Digested DNA-samples are separated in a 0.7% agarose (Promega) gel overnight at 15 mA current and transferred to positively charged nylon membrane (Boehringer Mannheim) using vacuum blotter. RNA probes are synthesized using T3 RNA polymerase on the pBluescript vector carrying hydrophobin gene sequence and labeled with digoxigenin-11-UTP. The membrane is hybridized and developed according to the supplier's instructions (Boehringer Mannheim, The DIG user's guide for filter hybridization). The membrane is pre-hybridized at 50° C. for 2 h and hybridized at 50° C. in a “DIG EasyHyb” hybridization solution (Boehringer Mannheim) overnight with a digoxigenin-UTP labelled RNA probe. The concentration of RNA probe is 100 ng/ml. After hybridization the membrane is washed in SSC buffers, blocked and detected using “Anti-Digoxigenin-AP alkaline phosphatase (Boehringer Mannheim). Chemiluminescent detection is done with CSPD-substrate and the membrane is exposed to X-ray film (Boehringer Mannheim). Presence of the transgene insertion is proved in comparison to DNA of non-transgenic Camelina sativa-plant DNA as negative control, and to plasmid DNA carrying the gene sequence mixed with non-transgenic plant DNA as positive control.

Based on the Southern blot analysis, plants with a desired copy number may be selected and crossed with each other to obtain higher copy number and accordingly a higher expression of the heterologous gene product.

### Example 12

### Agrobacterium Mediated Transformation with Mouse Anti-CEA Single Chain Fab Fragment

Synthetic sequence for part of Arabidopsis thaliana seed storage albumin 2 signal peptide (2S2 SP) coded by nucleotides 80-118 of Genebank accession number NM_{—}118849 and mouse anti-CEA single chain fab fragment (aCEA-Fv) (UniProt accession number Q921A6) (SEQ ID NO: 20 for synthetic gene, SEQ ID NO:21 for amino acid sequence) is obtained from GeneScript. The 2S2-aCEA-Fv is amplified by PCR from GeneScripts vector pUC57 using primers o115 (SEQ ID NO: 14) and aCEA-R (SEQ ID NO: 22). 6× His-KDEL-Stop-fragment is made by PCR using two overlapping primers aCEA-His-F (SEQ ID NO: 23) and o118 (SEQ ID NO:16). 2S2-aCEA-Fv and 6× His-KDEL-Stop-fragment have homologous overlap and are joined together in second PCR using primers o115 and o118. In addition to homology to aCEA-Fv, 6× His-KDEL-Stop-fragment contain homology to arcelin 3′UTR.

Plant expression vector pA14-PPhas-2S2-Tarc-2-3 contain Phaseolus vulgaris phaseolin promoter (SEQ ID NO: 1), Arabidopsis thaliana seed storage albumin 2 signal peptide (2S2 SP) (SEQ ID NO:10) and arcelin 3′UTR (SEQ ID NO:4). pA14-PPhas-2S2-Tarc-2-3 is cut with restriction enzymes XhoI and Paul between the 2S2 SP and arcelin 3′UTR and joined via end-homologies with 2S2-ELN-6× His-KDEL-Stop fragment using Gibson Assembly reaction mix of New England Biolabs.

What is claimed is:

1. A method to produce one or more heterologous gene products in dicotyledonous plant seeds, said method comprising the steps of:
a) providing a haploid cell culture;
b) obtaining haploid embryos or callus from the haploid cell culture;
c) regenerating the embryos or the callus to double haploid plantlets with shoots and roots;
d) obtaining double haploid plants from the doubled haploid plantlets;
e) obtaining double haploid seeds from the mature double haploid plants;
f) surface sterilizing the double haploid seeds;
g) germinating the seed of step f) in vitro to form doubled haploid plantlets or sprouts;
h) transforming explants from the double haploid plantlets or sprouts with Agrobacterium tumefaciens carrying at least one vector comprising a nucleotide sequence encoding the heterologous gene product under a seed specific promoter;
i) regenerating the transformed explants to transgenic doubled haploid plants; and
j) allowing the plants to produce seeds expressing the heterologous gene product.

2. The method of claim 1, wherein the plant is Camelina sativa.

3. The method of claim 2, wherein the seed specific promoter is napin promoter.

4. The method of claim 1, wherein the heterologous gene product is selected from the group consisting of lauric acid-acyl carrier protein, hydrophobin, tropoelastin, an antibody, and a growth factor.

5. A clonal-like isogenic production system in dicotyledonous plant seeds to produce heterologous gene products in large quantities with high uniformity of the product, said system comprising:
a) a haploid cell culture to provide haploid callus or embryos;
b) a protocol to regenerate the haploid callus or embryos to doubled haploid plantlets;
c) a protocol to grow doubled haploid plants from the plantlets of step b);
d) a protocol to produce double haploid seeds of plants of step c);
e) a protocol to produce explants from the double haploid seeds of step d)
f) a protocol to transform a nucleotide sequence encoding the heterologous gene product under a seed specific promoter to the explants of step e);
g) a protocol to regenerate the explants to doubled haploid transgenic plants;
h) a protocol to grow the doubled haploid transgenic plants to maturity, and to collect doubled haploid transgenic seeds of the mature plants; and
i) a protocol to harvest the heterologous gene products from the transgenic seeds

6. The clonal-like production system of claim 5, wherein the haploid cell culture is a Camelina sativa microspore culture.

7. The clonal-like production system of claim 5, wherein the seed specific promoter is napin promoter.

8. The clonal-like production system of claim 7, wherein the heterologous gene product is selected from the group consisting of lauric acid-acyl carrier protein, hydrophobin, tropoelastin, an antibody, and a growth factor.

9. The clonal-like production system of claim 7, wherein the transgenic seeds are sprouted and the gene product is collected from the sprouted seeds.

10. A method to control production of heterologous gene products in dicotyledonous plant seeds, said method comprising the steps of:
a) providing a haploid cell culture;
b) obtaining haploid embryos or callus from the haploid cell culture;
c) regenerating the embryos or the callus to double haploid plantlets with shoots and roots;
d) obtaining double haploid plants
e) obtaining double haploid seeds from the double haploid plants
f) surface sterilizing the seed of step e)
g) germinating the seeds of step f) in vitro
h) transforming explants from double haploid plantlets or sprouts with Agrobacterium tumefaciens carrying at least one vector comprising a nucleotide sequence encoding a nucleotide sequence encoding the heterologous gene product under a seed specific promoter;
i) regenerating the transformed explants to transgenic doubled haploid plants;
j) determining copy number of the nucleotide sequence encoding the heterologous gene product in several doubled haploid plants;
k) selecting lines with preferred copy numbers, and
l) allowing the selected lines to produce flowers and crossing the lines, thereby receiving next generation with a increased copy number and desired level of production of the heterologic gene product.

11. The method of claim 10, wherein the haploid cell culture is Camelina sativa microspore culture.

12. The method of claim 11, wherein the seed specific promoter is napin-promoter.

13. The method of claim 12, wherein the heterologous gene product is selected from the group consisting of lauric acid-acyl carrier protein, hydrophobin, tropoelastin, an antibody, and a growth factor.

14. A transgenic doubled haploid seed of Camelina sativa expressing a heterologous gene product.

15. A double haploid dicotyledonous clonal-like production system comprising:
an isogenic doubled haploid plant line for production of seeds;
wherein the seeds are germinated and grown to flowering plants, the flowering plants are transformed with Agrobacterium-mediated flowerdip transformation to express one or more heterologous gene products, and the resulting transgenic plants are grown and allowed to produce seeds expressing the heterologous gene product.


SEQUENCE LISTING (26 sequences)

SEQ ID NO: 1 - DNA, 1470 bp, Phaseolus vulgaris
  promoter (1)..(1470): phaseolin promoter
aattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataatgatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatgatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataagatttatggtggactaattttcatatatttcttattgcttttaccttttcttggtatgtaagtccgtaactagaattactgtgggttgccatggcactctgtggtcttttggttcatgcatggatgcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttc

SEQ ID NO: 2 - DNA, 654 bp, artificial sequence
  chemically synthesized
atgggattcgtccttttcagccagcttccttctttcctcttggtgtctaccctcctcttgttcctcgtgatctctcactcttgcagggccatgaacatccagaccagaggtaagaagattatctacggaagcctccctaggtacaagtctccatctcctgctcacagcttctctagcagaactcctcagttcaactaccctgctctccctagcttcgctatccttccttacaacctcctcgctatgttcgctagactccctgttgttttcctctacgctttcgtggctttcggtgctcttgttgctgctctccctggtggacatcctggaactactactcctcctgttaccaccactgttaccgttactacccctccatctaccactactatcgctgctggtggaacttgcactaccggatctttgtcttgctgcaaccaggtgcagtctgcttcttcttctcctgttactgctctccttggactcctcggaatcgttctctctgatctcaacgttctcgtgggaatctcttgctctcctctcaccgttatcggagttggaggatctggatgttctgctcagactgtttgctgcgagaacacccagttcaacggactcatcaacatcggatgcaccccaatcaacatcctctga

SEQ ID NO: 3 - protein, 217 aa, artificial sequence
  chemically synthesized
MGFVLFSQLPSFLLVSTLLLFLVISHSCRAMNIQTRGKKIIYGSLPRYKSPSPAHSFSSRTPQFNYPALPSFAILPYNLLAMFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 4 - DNA, 1280 bp, artificial sequence
  chemically synthesized
actcccaaaaccaccttccctgtgacagttaaaccctgcttatacctttcctcctaataatgttcatctgtcacacaaactaaaataaataaaatgggagcaataaataaaatgggagctcatatatttacaccatttacactgtctattattcaccatgccaattattacttcataattttaaaattatgtcatttttaaaaattgcttaatgatggaaaggattattataagttaaaagtataacatagataaactaaccacaaaacaaatcaatataaactaacttactctcccatctaatttttatttaaatttctttacacttctcttccatttctatttctacaacattatttaacatttttattgtatttttcttactttctaactctattcatttcaaaaatcaatatatgtttatcaccacctctctaaaaaaaactttacaatcattggtccagaaaagttaaatcacgagatggtcattttagcattaaaacaacgattcttgtatcactatttttcagcatgtagtccattctcttcaaacaaagacagcggctatataatcgttgtgttatattcagtctaaaacaattgttatggtaaaagtcgtcattttacgcctttttaaaagatataaaatgacagttatggttaaaagtcatcatgttagatcctccttaaagatataaaatgacagttttggataaaaagtggtcattttatacgctcttgaaagatataaaacgacggttatggtaaaagctgccattttaaatgaaatatttttgttttagttcattttgtttaatgctaatcccatttaaattgacttgtacaattaaaactcacccacccagatacaatataaactaacttactctcacagctaagttttatttaaatttctttacacttcttttccatttctatttctatgacattaactaacatttttctcgtaattttttttcttattttctaactctatccatttcaaatcgatatatgtttatcaccaccactttaaaaagaaaatttacaatttctcgtgcaaaaaagctaaatcatgaccgtcattttagcattaaaacaacgattcttgtatcgttgtttttcagcatgtagtccattcttttcaagcaaagacaacagctatataatcatcgtgttatattcagtctaaaacaacagtaatgataaaagtcatcattttaggcctttctgaaatatatagaacgacattcatggtaaaaaatcgtcattttagatcc

SEQ ID NO: 5 - DNA, 2037 bp, artificial sequence
  chemically synthesized
tgcgcaactttcgccctctgcttcctcctcaccaacgccggaggtgtaccaggagccattcctggtggagttcctggaggtgtattttatccaggagctggtcttggagcacttggaggaggcgcactaggtccaggtggaaaacctcttaaaccagttcctggaggacttgcgggtgctggtcttggagcaggtctcggtgcattcccagcagttacatttccaggagctctagtacctggtggagtagcagacgctgctgcagcttataaagctgctaaggctggtgctggtcttggtggagtcccaggagttggtggattaggagtatctgcagctccatctgtaccaggtgcagtagttcctcaacctggagcaggagttaaacctggaaaagtaccaggtgtaggtctaccaggagtatacccaggtggtgtactaccaggagctagattcccaggtgtaggagtgctacctggagttccaactggtgcaggagttaagcctaaggctccaggtgtaggtggagcttttgctggaattcctggagttggaccatttggtggtccacaaccaggagttccactaggataccctattaaagcccccaaactacctggtggatatggactaccatacacaacagggaaactaccatatggttatggtcctggaggagtagctggtgcagctggaaaagctggttacccaacaggtacaggtgttggcccacaagcagcagctgcagcggcagctaaagcagcggcaaagttcggtgctggagcagccggagtcctcccaggtgttggaggtgctggtgttcctggagtgccaggagcaattcctggaattggaggtatcgcaggtgttggaactccagctgccgctgcagctgcggcagcagcagctaaagcagccaagtatggagctgctgcaggattagtacctggtggaccaggctttggaccaggagtagttggtgttccaggagctggagttccaggtgttggtgtcccaggagctggtattccagtcgttcctggtgctgggattccaggtgctgcggttccaggtgttgtgtcaccagaagcagctgctaaggcagctgcaaaggcagctaaatacggagcaagacccggagttggagttggaggcattcctacttacggagttggagctggaggctttccaggctttggtgttggagttggaggaatccctggagtggcaggtgttcctggtgttggaggtgttccaggagttggaggtgtcccaggagttggtatttcccccgaagctcaagcagcagctgctgccaaagctgctaagtacggtttagtccctggtgttggagtagctcctggagttggtgtagctcctggtgtcggtgtggctccaggagttggattggctccaggagttggtgtagctccaggagtaggtgtagctcctggcgttggagtagctccaggcattggacctggtggagttgcagctgcagcaaaatccgctgccaaggtagctgctaaagcacaacttagagctgcagctgggcttggtgctggaatccctggacttggagttggtgttggcgttccaggacttggagttggtgctggtgttcctggacttggagttggtgctggtgttcctggtttcggagcagtacctggagccctagctgcagctaaagcagccaaatatggagcagcagtacctggtgttcttggaggtctcggagctctcggtggagtaggcattccaggaggtgtagttggagctggaccagcagctgcagctgccgcagctaaagctgctgcaaaagccgcgcaatttggtctagtaggagctgctggactaggaggactaggagtcggaggtcttggagttccaggtgttggaggccttggaggtatacctccagctgcagctgctaaagcagctaaatacggagtagcagcaagaccaggtttcggattgtctccaattttcccaggtggtgcctgcctagggaaagcttgtggcagaaagagaaaa

SEQ ID NO: 6 - protein, 679 aa, artificial sequence
  chemically synthesized
CATFALCFLLTNAGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAAPSVPGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPGVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 7 - DNA, 1145 bp, Brassica napus
  promoter (1)..(1145): pNap promoter
aagctttcttcatcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaagtggcataccgttctcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacga

SEQ ID NO: 8 - DNA, 1432 bp, artificial sequence
  chemcially synthesized
gaagacacaatggccaccacctctttagcttccgctttctgctcgatgaaagctgtaatgttggctcgtgatggccggggcatgaaacccaggagcagtgatttgcagctgagggcgggaaatgcgccaacctctttgaagatgatcaatgggaccaagttcagttacacggagagcttgaaaaggttgcctgactggagcatgctctttgcagtgatcacaaccatcttttcggctgctgagaagcagtggaccaatctagagtggaagccgaagccgaagctaccccagttgcttgatgaccattttggactgcatgggttagttttcaggcgcacctttgccatcagatcttatgaggtgggacctgaccgctccacatctatactggctgttatgaatcacatgcaggaggctacacttaatcatgcgaagagtgtgggaattctaggagatggattcgggacgacgctagagatgagtaagagagatctgatgtgggttgtgagacgcacgcatgttgctgtggaacggtaccctacttggggtgatactgtagaagtagagtgctggattggtgcatctggaaataatggcatgcgacgtgatttccttgtccgggactgcaaaacaggcgaaattcttacaagatgtaccagcctttcggtgctgatgaatacaaggacaaggaggttgtccacaatccctgacgaagttagaggggagatagggcctgcattcattgataatgtggctgtcaaggacgatgaaattaagaaactacagaagctcaatgacagcactgcagattacatccaaggaggtttgactcctcgatggaatgatttggatgtcaatcagcatgtgaacaacctcaaatacgttgcctgggtttttgagaccgtcccagactccatctttgagagtcatcatatttccagcttcactcttgaatacaggagagagtgcacgagggatagcgtgctgcggtccctgaccactgtctctggtggctcgtcggaggctgggttagtgtgcgatcacttgctccagcttgaaggtgggtctgaggtattgagggcaagaacagagtggaggcctaagcttaccgatagtttcagagggattagtgtgatacccgcagaaccgagggtgtaactaatgaaagaagcatctgttgaagtttctcccatgctgttcgtgaggatactttttagaagctgcagtttgcattgcttgtgcagaatcatggtctgtggttttagatgtatataaaaaatagtcctgtagtcatgaaacttaatatcagaaaaataactcaatgggtcaaggttatcgaagtagtcatttaagctttgaaatatgttttgtattcctcggcttaatctgtaagctctttctcttgcaataaagttcgcctttcaatgggccc

SEQ ID NO: 9 - DNA, 63 bp, Arabidopsis thaliana
  sig_peptide (1)..(63): seed storage albumin 2 signal peptide (2S2 SP)
atggcaaacaagctcttcctcgtctgcgcaactttcgccctctgcttcctcctcaccaacgcc

SEQ ID NO: 10 - DNA, 57 bp, artificial sequence
  chemcially synthesized
gttggaaatacataatctagacgtttaaacccgggaattcattgtactcccagtatc

SEQ ID NO: 11 - DNA, 42 bp, artificial sequence
  chemically synthesized
cccatgatcatgcattcagaaagaagtgagtgatattagagg

SEQ ID NO: 12 - DNA, 20 bp, artificial sequence
  chemically synthesized
actcccaaaaccaccttccc

SEQ ID NO: 13 - DNA, 53 bp, artificial sequence
  chemcially synthesized
gactggacagctgcggaccgggcccggatctaaaatgacgattttttaccatg

SEQ ID NO: 14 - DNA, 21 bp, artificial sequence
  chemically synthesized
ctgcgcaactttcgccctctg

SEQ ID NO: 15 - DNA, 25 bp, artificial sequence
  chemically synthesized
ttttctctttctgccacaagctttc

SEQ ID NO: 16 - DNA, 67 bp, artificial sequence
  chemically synthesized
gaaagcttgtggcagaaagagaaaacatcaccatcaccatcacaaggatgagctttgacactcccaa

SEQ ID NO: 17 - DNA, 55 bp, artificial sequence
  chemcially synthesized
agcagggtttaactgtcacagggaaggtggttttgggagtgtcaaagctcatcct

SEQ ID NO: 18 - DNA, 50 bp, artificial sequence
  chemically synthesized
cacacccgggaagctttcttcatcggtgattgattcctttaaagacttat

SEQ ID NO: 19 - DNA, 36 bp, artificial sequence
  chemcially synthesized
cattcgtgtatgtttttaatcttgtttgtattgatg

SEQ ID NO: 20 - DNA, 723 bp, artificial sequence
  chemically synthesized
caggttaagctccaacaatctggacctgagcttaagaaacctggtgagactgtgaagatcagctgcaaggcttctggatacaccttcaccgactacggaatgaactgggttaagcaggctcctggaaaaggacttaagtggatgggatggatcaacacctacactggtgagcctacctacgctgatgatttcaagggaaggttcgctttcagcctcgagacttctgcttctaccgcttacctccagatcaacaacctcaagaacgaggacaccgccacctatttctgcgctagaaaggatctcctccgttacttcgattactggggacagggaactaccgttaccgtttcttctggtggtggtggaagcggaggtggtggatcaggtggtggtggttctgatatcgagcttacccagtctccttctagcctctctgcttctctcggaggaaaggttaccatcacttgcaaggctagccaggacatcaacaagtacattgcctggtatcagcacaagcctggaaagggtcctagatctgctcacacccttcacatctacatccagcctggaatcccttctaggttctctggatctggaagcggaagggactactctttcagcatctctaacctcgagcctgaggatatcgctacctactactgcctccactacgataaccttcacaccttcggaggtggaactaagctcgaacttaagagggct

SEQ ID NO: 21 - protein, 361 aa, artificial sequence
  chemcially synthesized
QVKLQQSGPELKKPGETVKISCKASGYTFTDYGMNWVKQAPGKGLKWMGWINTYTGEPTYADDFKGRFAFSLETSASTAYLQINNLKNEDTATYFCARKDLLRYFDYWGQGTTVTVSSGGGGSGGGGSGGGGSDIELTQSPSSLSASLGGKVTITCKASQDINKYIAWYQHKPGKGPRSAHTLHIYIQPGIPSRFSGSGSGRDYSFSISNLEPEDIATYYCLHYDNLHTFGGGTKLELKRAIDGRAAAAAAAGGAGQGGYGGLGSQGAGRGGQGAGIDGRQPLEDRSNGNGNVCPPGLFSNPQCCATQVLGLIGLDCKVPSQNVYDGTDFRNVCAKTGAQPLCCVAPVAGQALLCQTAVGA

SEQ ID NO: 22 - DNA, 23 bp, artificial sequence
  chemcially synthesized
agccctcttaagttcgagcttag

SEQ ID NO: 23 - DNA, 65 bp, artificial sequence
  chemically synthesized
ctaagctcgaacttaagagggctcatcaccatcaccatcacaaggatgagctttgacactcccaa

SEQ ID NO: 24 - DNA, 35 bp, artificial sequence
  chemically synthesized
tgaatgcatgatcatgggattcgtccttttcagcc

SEQ ID NO: 25 - DNA, 41 bp, artificial sequence
  chemically synthesized
ggaaggtggttttgggagttcagaggatgttgataggggtg

SEQ ID NO: 26 - DNA, 13 bp, artificial sequence
  chemically synthesized
tgaatgcatgatc